(19) United States
(12) Patent Application Publication
(10) Pub. No.: US 2004/0161861 A1
(43) Pub. Date: Aug. 19, 2004

(54) Glycoconjugate sensors

(76) Inventors: Kalle Levon, New York, NY (US); Olga Tarasenko, Brooklyn, NY (US); Bin Yu, West Hartford, CT (US)

Correspondence Address: STRAUB & POKOTYLO, 620 TINTON AVENUE, BLDG. B, 2ND FLOOR, TINTON FALLS, NJ, 07724, US

(21) Appl. No.: 10/719,688

(22) Filed: Nov. 21, 2003

Related U.S. Application Data
(60) Provisional application No. 60/428,067, filed on Nov. 21, 2002.

(51) Int. Cl. G01N 33/543

(52) U.S. Cl. 436/518

(57) ABSTRACT

A glycoconjugate sensor specific to a target biological entity may be fabricated by coating a support surface with polymers appended with carbohydrates. The carbohydrates appended to the polymers may be chosen by (i) identifying the surface glycoconjugates of a target biological entity and (ii) selecting corresponding carbohydrates that may specifically bind with the identified TAMPs, such as glycoconjugates. An ELISA platform may be used as the glycoconjugate sensor for detecting specific carbohydrate binding of the sensor to spores.

### § 0.1 RELATED APPLICATIONS

This application claims the benefit of U.S. Provisional Application Serial No. 60/428,067, titled “GLYCONCONJUGATE SENSORS,” filed on Nov. 21, 2002 and listing Kalle Levon, Olga Tarasenko and Bin Yu as the inventors. That application is expressly incorporated herein by reference.

### § 0.2 GOVERNMENT FUNDING

This invention was made with Government support and the Government has certain rights in the invention as provided for by contract number 0660076225 awarded by DARPA.

### §1. BACKGROUND

§ 1.1 Field of the Invention

The present invention concerns sensor development and target molecule recognition in general. In particular, the present invention concerns developing and using sensors to detect biological molecules, such as bacterial spores, using monovalent, polyvalent, or multivalent carbohydrate interactions with target-associated molecular patterns (TAMPs), such as glycoconjugates for example, on the surface of the target molecules.

§ 1.2 Related Art

Although carbohydrates are ubiquitous in both prokaryotic and eukaryotic cells, an appreciation of their varied functions is only beginning to emerge. (See, e.g., A. Varki, Glycobiology, 1993, 3, 97-130.) The surfaces of mammalian and bacterial cells are decorated with complex carbohydrates that exist as glycoconjugates such as glycoproteins, glycolipids, glycosaminoglycans and proteoglycans. For a number of genera, including Bacillus, the presence of extracellular polysaccharides in spores has been demonstrated. (See, e.g., Marz et. al, Bacteriol., 1970, 101, 196-201; Scherer and Somerville, Eur. J. Biochem., 1977, 72, 479-90; Warth et. al, J. Cell Biol., 1963, 16, 578-592; Kornberg et. al, M. Ann. Rev. Biochem., 1968, 37, 51-78; and Hiragi, J. Gen, Microbiol., 1972, 72, 87-99.) In biological systems, carbohydrates are often associated with specific recognition and signaling processes leading to important biological functions or diseases, including adhesion, cell-cell recognition, activation, sporulation (in the Bacillus genus), and maturation of living organisms. (See, e.g., Crocker and Feizi, Curr. Opin. Struct. Bio., 1996, 6, 679-691; Feizi, T., Immunol. Rev., 2000, 173-79-88; and Feizi, T., Glycoconj. J., 2000, 17, 553-565.) More specific and crucial biological roles of oligosaccharides are either mediated by oligosaccharide sequences, by common terminal sequences, or even by further modifications of the sugars themselves. However, such oligosaccharide sequences are also more likely to be targets for recognition by pathogenic toxins and microorganisms (See, e.g., Varki, A., Glycobiology, 1993, 3, 97-130; and Hirmo et. al, Analytical Biochem. 1998, 257, 63-66.) or other molecules. Many of a host's receptors for microbes are glyconjugates. (See, e.g., Karlsson, et. al, APMIS Suppl., 1992, 27, 71-83.) Conversely, carbohydrates and other microbe-derived molecules may serve as main antigenic structures that host receptors recognize in host-pathogen interactions. (See, e.g., Wang, D., and Kabat, E. A., In Structure of antigens, Regenmortal M. H. V. V., Ed.; CRC Press, Boca Raton, Fla.; 1996; Vol. 3, 247-276.)

Glycoconjugates have been extensively used for studying carbohydrate binding sites in histochemical and cytochemical experiments. (See, e.g., Kayser, et. al, Eur. J. Cancer, 1994, 30A, 653-657; Kayser, et. al, J. Analyt. Quant. Cytol, Histol., 1995, 17, 135-142; Bovin, et. al, Glycocon. J., 1995, 12, 427; Rye, P. D., and Bovin, N. V. Glycobiology, 1977, 7, 179-82; Leteux, et. al, Glycobiology, 1998, 8. 227-236; and Houseman, B. T., and Mrksich, M., Topics Current Chem., 2002, 218, 1-44.) Their ability to bind carbohydrate epitopes was established for a number of important cell surface proteins, such as clusters of differentiation and adhesion factors. Furthermore, practically all cells investigated with glycoconjugates probes showed the ability to selectively bind mono- or oligosaccharides (See, e.g., Danguy, et. al, Trends Glycosci. Glycotechnol., 1995, 36, 261-275.).

Bacillus cereus, Bacillus thuringiensis, and Bacillus subtilis are closely related pathogenic organisms that are, phenotypically or genotypically, difficult to differentiate. Spore forms of Bacillus are quite distinct, both morphologically and chemically. The spore's structure is rather sophisticated, and includes the following main parts: appendages, an exosporium, an outer coat, an inner coat, a cortex, and a core.

Spores have been examined in detail, with particular emphasis on the chemical composition of their appendages and exosporium. (See, e.g., Marz, et. al, J. Bacteriol., 1970, 101, 196-201; and Scherer, P. S., and Somerville, H. J., Eur. J. Biochem., 1977, 72, 479-90.) It has been recently reported, for example, that B. cereus spores' structure resembled a nap with hair-like projections known as an filamentous appendages or pilus-like structures (See, e.g., Gerhardt, P., and Ribi, E., J. Bacteriol., 1964, 88, 1774-1789.) originating from an exterior basal membrane of the exosporium. (See, e.g., Hachizuka, et. al, J. Bacteriol., 1966, 91: 2382-2384; Sousa, et. al, Nature, 1976, 263, 53-54; and Hultgren, et. al, S. Cell, 1993, 73, 887-901.) Filamentous or pilus-like structures are present on spores that are a variety of Bacillus, although such structures have not been observed in B. subtilis to date. (See, e.g., Kozuka, S., and Tochikubo, K., Microbiol. Immunol., 1985, 29, 21-37; and Driks, A., Microbiol. Molecul. Biol. Rev., 1999, 63, 1-20.) B. cereus appendages contain a very high concentration of proteins, followed by carbohydrates. However, fewer lipids are present (See, e.g., Marz, et. al, J. Bacteriol., 1970, 101, 196-201.) The exosporium of B. cereus spores consists mainly of proteins (52%), amino and neutral polysaccharides (20%), and lipids (18%). (Glucose and rhamnose are considered the principal neutral sugars (See, e.g., Scherer, P. S., and Somerville, H. J., Eur. J. Biochem., 1977, 72, 479-90.).) Biochemical experiments demonstrated that the coat of B. subtilis is largely composed of proteins with minor amounts of carbohydrates and lipids. (See, e.g., Warth, et. al , J. Cell Biol., 1963, 16, 578-592; Kornberg, et. al, Ann. Rev. Biochem., 1968, 37, 51-78; and Hiragi. Y., J. Gen, Microbiol., 1972, 72, 87-99.)

Considerable efforts have been directed towards understanding and mimicking surface carbohydrate recognition, as well as developing an effective system to control the recognition processes. Determining which carbohydrate epitopes are indispensable for specific recognition events remains a challenge. Spore-specific carbohydrate binding markers of Bacillus species have not yet been previously described. The problem of identifying specific carbohydrates for a species is compounded by the diversity of carbohydrate structures and the different contexts in which they occur. Moreover, carbohydrates often exhibit relatively low binding affinities for their binding partners. (See, e.g., Bertozzi, C. R., and Kiessling, L. L., Science, 2001; 291, 2357-2364.)

Therefore, there is a need to identify specific surface carbohydrates by which spores can be identified, as well as sensoring means to accurately distinguish spore species.

### § 3. SUMMARY OF THE INVENTION

The present invention uses how carbohydrates function as recognition signals for purposes of sensor development. The present invention may use carbohydrate binding interactions, such as carbohydrate-carbohydrate binding, as a basis to create a sensor for detecting biological entities. Thus, the present invention may use interactions of carbohydrates as a means to identify specific biological molecules, such as spores.

Target-associated molecular patterns (TAMPs), such as carbohydrates or glycoconjugates for example, on the surface of a target biological entity can be identified and used to select carbohydrate binding components. The selected carbohydrate binding components are appended to polymers. The carbohydrate-appended polymers are used to coat a sensor's recognition surface. When exposed to a solution containing target biological entities, specific binding, such as carbohydrate-carbohydrate binding, may occur. The occurrence of such binding may be detected by a variety of means, such as colorimetry for example.

In one embodiment, the present invention overcomes the above-mentioned obstacles and elucidates carbohydrate interactions by using fluorescent labeled glycoconjugates as model systems to evaluate the mechanism of Bacillus spore recognition. However, any other type of transduction mechanism can be applied for the detection of the specific binding.

### § 4. BRIEF DESCRIPTION OF DRAWINGS

FIG. 1 is a scheme depicting the reduction of a protein's disulfide bonds by an excess of a sulfhydryl reagent (R—SH) such as 2% 2-mercaptoethanol.

FIGS. 2A and 2B are tapping mode of atomic force microscopy images of (A) Bacillus spores' isolated exterior layer including appendages after treatment with 2% 2-mercaptoethanol and (B) the spores' remaining inner parts. Height (a) and amplitude (b) are shown in the images.

FIG. 3 depicts the FACE method's fluorophore labeling reaction using NaBH₃CH as the reducing agent and AMAC to form the Schiff base.

FIG. 4 is an image of the polyacrylamide gel resulting from FACE analysis of glycoconjugates on an exterior of Bacillus spores. Lanes 1 and 10: AMAC-labeled monosaccharides standard; lanes 2-9: monosaccharide compositions of Bacillus spores' appendages'; lanes 2, 4, 6, and 8: monosaccharides from amino sugar hydrolysis; lanes 3, 5, 7, and 9: monosaccharides from neutral sugar hydrolysis; lanes 2 and 3: B. cereus' monosaccharide composition; lanes 4 and 5: B. thuringiensis' monosaccharides composition; lanes 6 and 7: B. subtilis' monosaccharides composition; lanes 8 and 9: B. pumilus' monosaccharides composition.

FIG. 5 shows the binding matches between spores (target entities) and immobilized glycoconjugates (ligands).

FIGS. 6 and 7 are graphs illustrating typical binding curves of target/s recognition.

### § 5. DETAILED DESCRIPTION

The present invention involves designing, fabricating and/or using glycoconjugate sensors to recognize, specifically, biological entities. The glycoconjugate sensor's recognition mechanism may use carbohydrate-carbohydrate interactions of glycoconjugate molecules—synthetic or natural—with the target biological entity. The glycoconjugate molecules may be provided on the substrate coating, or in solution with the sensor with the target biological entity.

In general, designing, fabricating and using the glycoconjugate sensor involves (i) identifying the target's surface TAMPs (e.g., glycoconjugates), (ii) identifying a carbohydrate binding partner to the identified surface TAMPs, (iii) fabricating a sensor with a coating of the carbohydrate binding partner appended to a polymer (functionalized so that the carbohydrate can be linked to it and having desired solubility properties) (referred to as a “ligand conjugate”) on a support surface, and (iv) exposing the sensor to a solution containing the targets to allow specific binding to take place.

Alternatively, the carbohydrate component in the ligand conjugate can be selected using high throughput methods, such as flow cytometry for example, or other screening methods for selective binding.

The following description is presented to enable one skilled in the art to make and use the invention, and is provided in the context of particular embodiments and methods. Various modifications to the disclosed embodiments and methods will be apparent to those skilled in the art, and the general principles set forth below may be applied to other embodiments, methods, and applications. Thus, the present invention is not intended to be limited to the embodiments and methods shown and the inventors regard their invention as the following disclosed methods, apparatus, and materials and any other patentable subject matter to the extent that they are patentable.

In the following, methods to determine surface TAMPs (e.g., glycoconjugates) of a target (e.g., a target biological entity) are described in § 5.1. Sensor composition and fabrication, and sensor operation are explained in §§ 5.2 and 5.3, respectively. An exemplary embodiment of the present invention—a glycoconjugate sensor for spores—is described in § 5.4. Finally, some conclusions about the present invention are summarized in § 6.

§ 5.1 Determining Surface TAMPs, such as Glycoconjugates

The present invention exploits the ability of a carbohydrate ligand coupled to a sensor chip surface, defining the sensor's substrate, to specifically bind to TAMPs (e.g., glycoconjugates) on the target's surface to identify the target biological entity. Thus, unless such information is already known, a first step in designing a glycoconjugate sensor is identifying glycoconjugates on the target's surface and choosing corresponding carbohydrate binding partners to append to polymers and incorporate onto the sensor. Using spore surface analysis is not the only way to determine the glycoconjugate(s) (and therefore select a carbohydrate binding partner(s). For example, glycoconjugate(s) can also be identified, and binding partners selected, using a random set of glycomolecules in a screening experiment for the specific binding.

Surface glycoconjugates of the target may be identified by fluorophore assisted carbohydrate electrophoresis (“FACE”). Briefly, FACE analysis involves isolating carbohydrates from the target's surface and labeling them with fluorescent tags. The labeled carbohydrates may then be separated via polyacrylamide gel electrophoresis and identified by comparison to standards. Alternatively, the carbohydrate component in the ligand conjugate can be selected using high throughput methods, such as flow cytometry for example, or other screening methods for selective binding.

The binding partners (e.g., sugar molecules) can be conjugated with covalent linking, such as ester or amide bonding, or through ionic or other non-covalent interactions with the conjugating molecules which can be small molecular bifunctional or multifunctional linkers, or tethers, or dendrimers of various generations or synthetic or natural macromolecules of various molecular weights.

§ 5.2 Sensor Composition and Fabrication

The glycoconjugate sensor provides a mechanism for identifying target biological entities on a support surface coated with a substrate. The substrate includes carbohydrate binding partners, corresponding to the target's surface glycoconjugate(s) identified, appended to polymers which are coupled with a chip surface. Recall that the target's surface glycoconjugate(s) may have been previously identified, for examples as described in § 5.1.

A means of detecting a carbohydrate-carbohydrate binding match between the sensor and the target (such as calorimetric means) may be incorporated into the sensor. The bindings used for recognition are not limited to carbohydrate-carbohydrate interactions, which may be used for the selection of specific sugar molecules. The glycoconjugates used in this invention can also interact with protein, lipid, or other sugar components on the surface of the target (e.g., spores).

§ 5.3 Operation of the Glycoconjugate Sensor

In general, sensor operation involves exposing the substrate-coated sensor surface to a solution containing target biological entities. Note, however, that the binding can also occur in a solution if the method of detection is a solution-based method such as flow cytometry.

The way in which the sensor is used to detect a carbohydrate binding with the spores depends on the type of sensor used. For example, sensors which incorporate a colorimetric detection system reveal binding matches by their degree of color change. Alternatively, the support surface of the sensor could be: a plate for surface acoustic wave measurement; a quartz crystal microbalance, or some other surface which is sensitive to changes in mass; any electrochemical device such as on ion sensitive electrode or ion selective field effect transistor; a light emitting or otherwise optically active surface; etc.

§ 5.4 Exemplary Embodiment

In an exemplary embodiment of the present invention, target biological molecules are spores from the Bacillus genus, including B. cereus, B. thuringiensis, B. subtilis, and B. pumilus, and the glycoconjugate sensor is an ELISA assay. In the following, identification of surface carbohydrates is described in § 5.4.1. ELISA glycoconjugate sensor fabrication is then described in § 5.4.2. Thereafter, operation of the ELISA glycoconjugate sensor is described in § 5.4.3. Finally, spore detection and quantification is described in § 5.4.4.

§ 5.4.1 Identification of Surface TAMPs, such as Carbohydrates, using FACE

In general, identifying surface glycoconjugates of a target entity—in this case spores—includes (i) isolating spores' appendages and inner parts by cellular fractionation, (ii) visualizing the appendages and inner parts by atomic force microscopy, and (iii) performing FACE analysis on the isolated appendages.

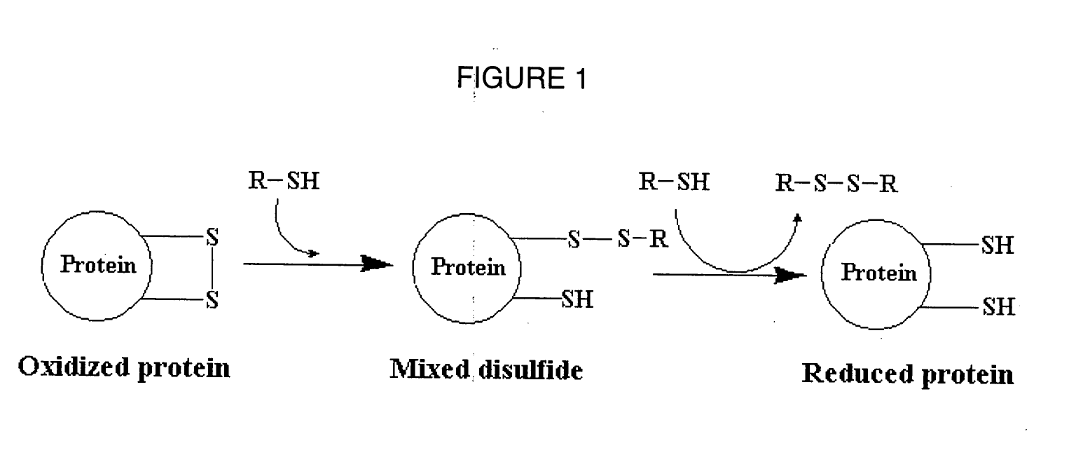

In this exemplary embodiment of the present invention, surface glycoconjugates are located on the spores' appendages. Thus, appendages were isolated from their respective spores by mixing 500 μL of a spore suspension (approximately 2×10⁶ spores) with 2% 2-mercaptoethanol (1 mM carbonate-bicarbonate buffer, pH 10.0) (See FIG. 1) and incubating for 2 hours at 37° C. as described in Kozuka, S., and Tochikubo, K., Microbiol. Immunol., 1985, 29, 21-37. After exposing the solution to the reagent, the mixture was centrifuged at 4,000×g for 20 minutes. The fractions, containing appendages (in the supernatant) and spores' inner parts (in the pellets) were washed with deionized water three-four times.

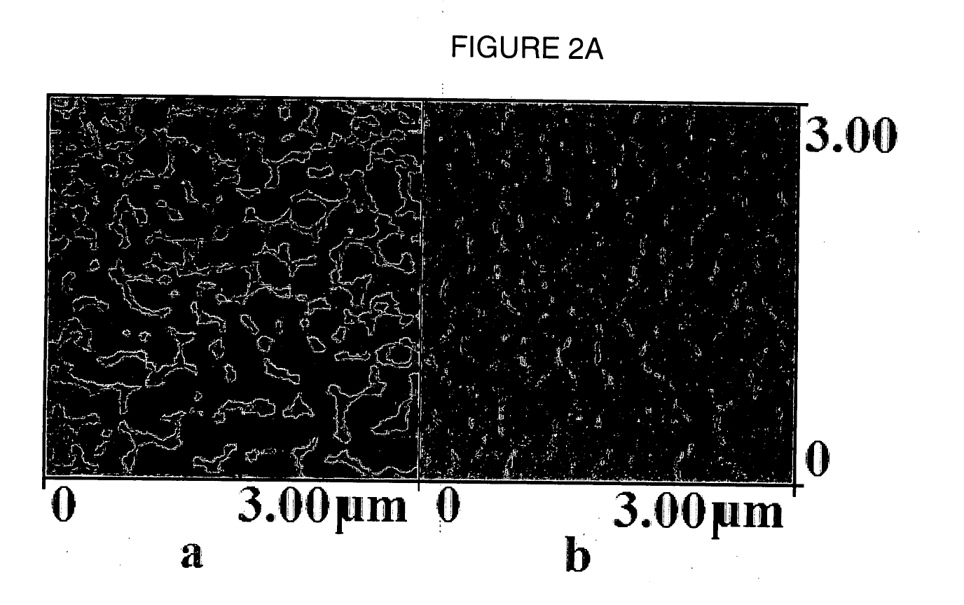

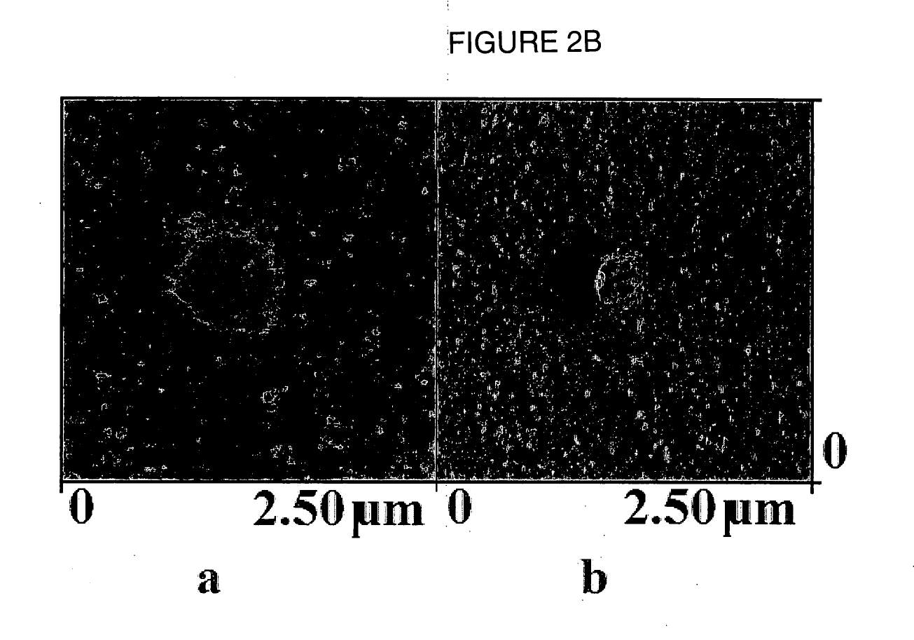

The isolated appendages and spores' inner parts of B. cereus, B. thuringiensis, B. subtilis, B. pumilus spores were examined with atomic force microscopy (AFM) imaging, as seen in FIGS. 2A and 2B. To prepare the bacterial spores' appendages for AFM, they were washed and immobilized on mica discs (from Digital Instruments, Inc., of Santa Barbara, Calif., USA) using sterile syringes. After drying in ambient air at room temperature, the prepared samples were mounted on an AFM sample holder for imaging. All AFM observations were carried out at room temperature (20° C.), using a Nano Scope® IIIa controller as well as a MultiMode™ microscope (from Digital Instruments, Inc.) operating in Tapping Mode (amplitude) together with an E scanner. A 125 μm silicon Nanoprobe (from Digital Instruments, Inc.) was employed as the cantilever/tip assembly. During tapping mode, the calculated spring constant was 0.3 N/m, the resonance frequency remained in the range of 240-280 kHz, the radius of curvature was approximately 10 nm, and the scan rate of was of 1 μm/s. The image data was flattened and high pass filtered to remove the substrate slope from images as well as high-frequency noise strikes, which were otherwise more pronounced in the high-resolution tapping mode imaging.

FIGS. 2A and 2B show the AFM image of isolated spore appendages and spores' inner parts, respectively. In both 2A and 2b, the left side (a) is the height and the right side (b) is the amplitude. The spores' relatively small diameter and peculiar shape seen in FIG. 2B indicate that they have lost their layer of appendages originating from the exosporium as compared to the morphology of spores without a 2% 2-mercaptoethanol treatment.

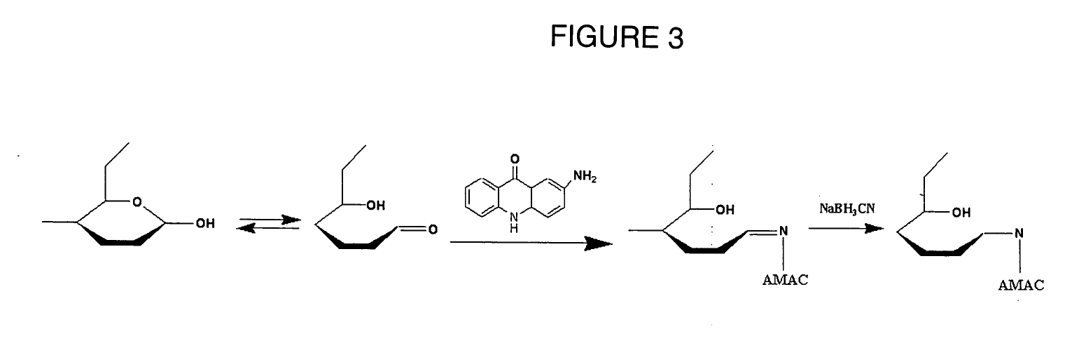

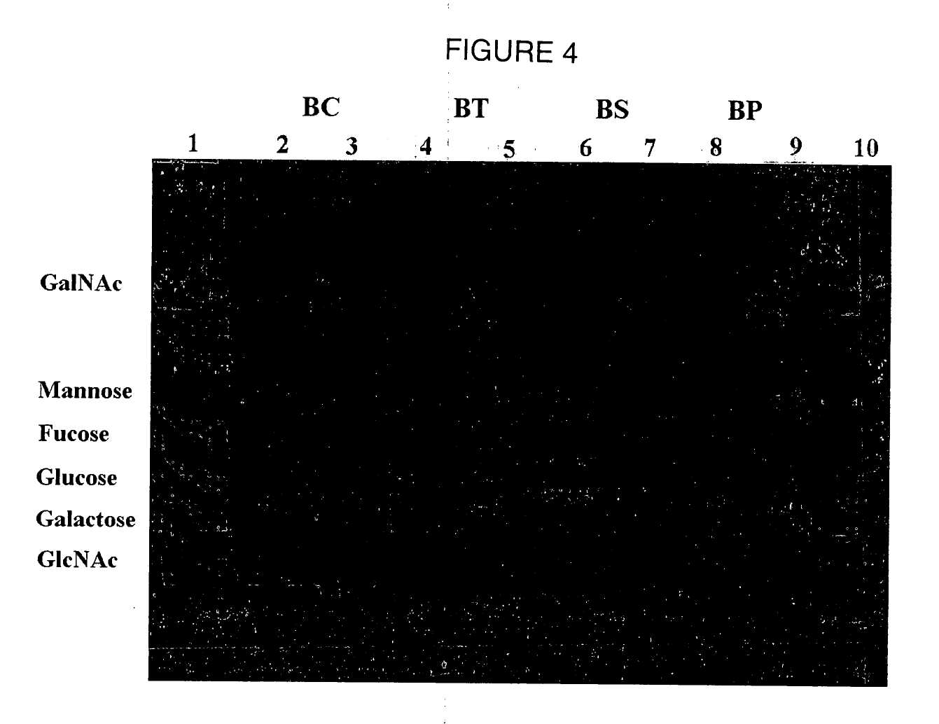

To identify the types of glycoconjugates on the surfaces of the spores' appendages, FACE was performed with FACE® monosaccharide composition kit, which allows analysis of both neutral and amine monosaccharides from intact glycoproteins, according to the manufacturer's instructions (from Glyko, Inc., Novato, Calif., USA). Briefly, monosaccharides were hydrolyzed from spores' appendages by dissolving in 2 M trifluoroacetic acid (TFA) at 100° C. for 5 hours if they were neutral, or by dissolving in 100 μl of 4 M HCl at 100° C. for 3 hours if they were amino sugars. After hydrolysis, the mixture was dried under reduced pressure. Dried monosaccharides from the amino hydrolysis reaction were re-N-acetylated by addition of a re-acetylation buffer solution. Dried monosaccharides from both hydrolyses were labeled with a fluorescent tag (AMAC), as shown in FIG. 3, and incubated overnight at 37° C. Fluorophore labeled monosaccharides were separated by polyacrylamide gel electrophoresis. (Electrophoresis was performed at 5° C. with a constant electric current per gel for 75 min.) The resulting band patterns, as shown in FIG. 4, represent the monosaccharide composition of the starting material. Standard mixtures of monosaccharides consisting of 100 pmol each of AMAC-labeled N-acetylglucosamine (GlcNAc), N-acetylgalactosamine (GalNAc), galactose, mannose, fucose, and glucose (lanes 1 and 10) were compared to the hydrolysis reactions (lanes 2-9) and used to identify their carbohydrates.

Spore appendages from the examined Bacillus species (B. cereus, B. thuringiensis, B. subtilis, and B. pumilus) exhibited unique carbohydrate profiles. Both B. cereus (lanes 2 and 3) and B. thuringiensis (lanes 4 and 5) contained neutral (even lanes) and amine (odd lanes) profiles. Galactose was identified in B. cereus spore's appendages through neutral and amine sugar profiles. B. thuringiensis's neutral sugar profile contained two monosaccharides—glucose and galactose—whereas its amine sugar profile contained galactose. Additional monosaccharides were detected on B. thuringiensis' appendages that could not be identified with certainty using the FACE® monosaccharide composition kit. In contrast to B. cereus and B. thuringiensis, B. subtilis (lanes 6 and 7) and B. pumilus (lands 8 and 9) spores' appendages exclusively exhibited neutral sugar profiles. Mannose, fucose, and galactose were detected in B. subtilis spores' appendages, while B. pumilus spores' appendages contained galactose and GlcNAc. Furthermore, appendages of B. cereus, B. thuringiensis, B. pumilus spores included both N-linked and O-linked oligosaccharides, whereas B. subtilis composed only O-linked oligosaccharides. (See, e.g., Tarasenko O., Islam Sh., and Levon K., “Monosaccharide and protein profiles analysis of the bacterial spores.” 226^{th} ACS National Meeting, New York, N.Y., Sep. 7-11, 2003. (abstract/oral); and Tarasenko O. M., Islam Sh., Alusta P., and Levon K. M., “Polyvalent ligand-receptor interactions for recognition of Bacillus spores,” 226^{th} ACS National Meeting, New York, N.Y., Sep. 7-11, 2003. (abstract/oral), both incorporated herein by reference).

Carbohydrates' determinants may serve as binding molecules and may be essential for recognition through the interaction between their carbohydrate moieties. (See, e.g., Siegelman, et. al, Cell, 1990, 61, 611; Iwabuchi, et. al, J. Biol. Chem., 1998, 273: 9130-9138; Handa, et. al, Methods Enzymol., 2000, 312, 447-458; Zheng. M., and Hakomori, S., Arch Biochem. Biophys., 2000, 374, 93-99; and Wang, et. al, Nat. Biotechnol. 2002, 20, 275-281.) Thus, profiling monosaccharides of oligosaccharides proved a powerful tool in differentiating Bacillus closely related species since FACE analysis determined the specific recognized carbohydrates epitopes of bacterial spores' appendages which service as receptors for recognition.

§ 5.4.2 Fabrication of ELISA Glycoconjugate Sensor for Spores

In general, ELISA glycoconjugate sensor fabrication includes (i) coating the wells of an ELISA plate with glycoconjugate-appended polymers, and (ii) washing and blocking.

In an exemplary embodiment of the present invention, the glycoconjugate sensor is an enzyme linked immunosorbent assay (“ELISA”) plate and binding matches were detected by colorimetric means. Fluoresceinated glycopolymers were coated as substrate subunits onto the wells of microtiter plates to capture target bacterial spores. Thus, using specific glycoconjugates as a capture reagent allowed spores to be used as ligands.

To fabricate the glycoconjugate sensor, the wells of an ELISA plate (Nunc, MaxiSorp) were coated with 20 μl multivalent glycoconjugate-PAA (polyacrylamide) overnight at 4° C. The plates were washed three times with 50 μl/well PBS containing 0.1% Tween-20. Blocking was accomplished by adding 12.5 μl/well of 3% BSA in PBS at 37° C. to the plates' wells and subsequently incubating for 1-2 hours at room temperature.

§ 5.4.3 Demonstration of Operation of ELISA Glycoconjugate Sensor for Spores

In general, operating an ELISA glycoconjugate sensor includes (i) incubating a spore solution in the ELISA plate wells, (ii) pre-complexing the glycoconjugate-spore complex (e.g., with anti-mouse(IgG+IgM)-horseradish peroxidase(HRP)-labeled conjugate), (iii) adding color substrate, and (iv) observing a colorimetric reaction. The exemplary ELISA glycoconjugate sensor was used by adding 7.5 μl of a spore solution to each well and incubating for 1-2 hours at room temperature with continuous shaking. After incubation, the plate was washed three times with PBS containing 0.1% Tween-20, after which 12.5 μl anti-mouse (IgG+IgM)-horseradish peroxidase (HRP)-labeled conjugate (1:4000 dilution) (Roche Diagnostics Corp., Indianapolis, Ind.) was added and incubated at 37° C. for 1 hour, thus pre-complexing the glycoconjugate-spore complex with the HRP-labeled secondary antibody conjugate. After washing again with the PBS solution, 2,2′-azino-bis(3-ethylbenzthiazoline-6-sulfonic acid)as liquid substrate system for ELISA was added to the wells. If the primary antibody, glycoconjugate, remains in a well, then the secondary antibody will bind to it and will also remain attached after washing. This allows carbohydrate-carbohydrate binding pairs to be visualized. The calorimetric reaction was terminated by addition of 12.5 μl/well of 1 M H₂SO₄.

Thus, microtiter-plate based ELISA provides a rapid method for evaluating carbohydrate-carbohydrate interactions using immobilized multivalent fluoresceinated polymers as a substrate for bacterial spore detection. The method was also used to detect carbohydrate-binding epitopes and to investigate the selectivity of glycoconjugates for various Bacillus spores.

§ 5.4.4 Spore Detection using the ELISA Glycoconjugate Sensor

In the exemplary embodiment of the present invention, spore capture by binding the sensor's substrate is evidenced by a colorimetric reaction with high optical density (OD). Thus, color change in the spore-containing solution signifies carbohydrate-carbohydrate binding matches between the sensor and target spores. Color intensity indicates the quantity of target bound to the sensor's substrate.

This test quantifies how much enzyme(HRP) is present by the amount of color produced. The more enzyme present, the more the HRP-labeled secondary antibody conjugate must be attached. The amount of secondary antibody present is determined by the amount of target available. Finally, because the first antibody such as glycoconjugates bind to antigen, the more antigen that is accessible, the more first antibody will be retained. The measure of color, therefore, reflects the amount of ligand-target initially present.

Colorimetric results may be measured by spectrophotometry. In an exemplary embodiment of the present invention, spectrophotometric measurements were carried out with a microplate reader SPECTRAmax®Plus 384 (from Molecular devices Corp., Sunnyvale, Calif., USA) at 405 nm. Blank readings were subtracted from the optical density of the final reaction to obtain the corrected absorbance value. To avoid simple experimental mistakes leading to incorrect results, it is recommended to conduct tests using duplicate (or, sometimes, more than two) samples and then calculate average data.

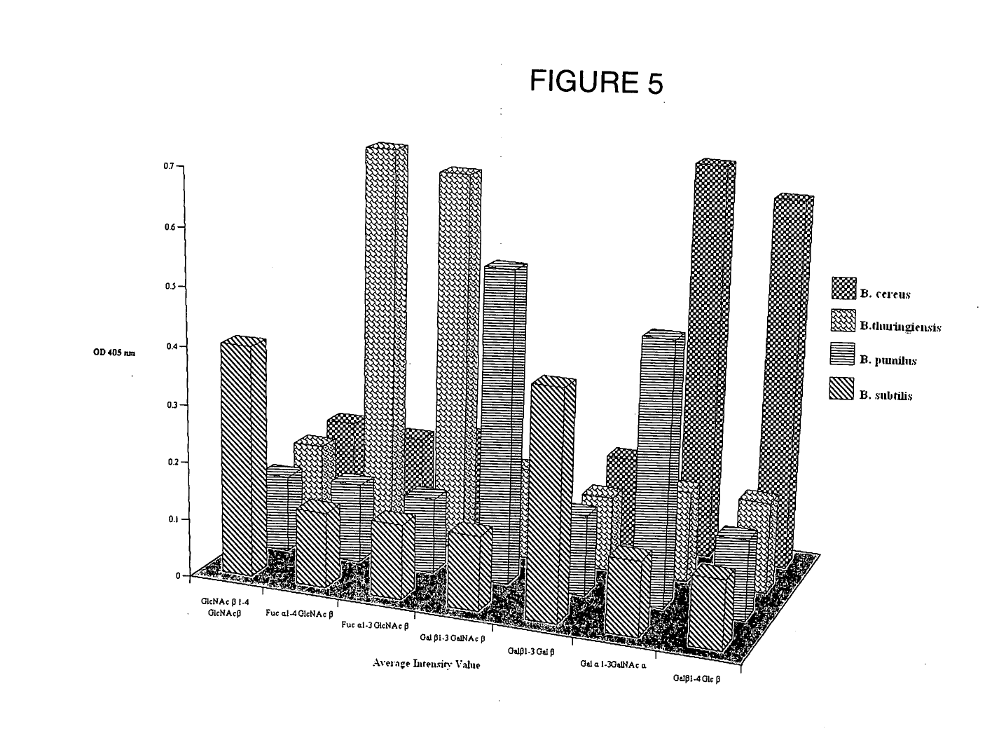

Certain glycoconjugates demonstrated selective affinity for different Bacillus related species, such as B. cereus, B. thuringiensis, B. subtilis and B. pumilus. These patterns presumably reflect a unique distribution of carbohydrate receptors at bacterial spore's appendages. The formation of a multivalent complex between glycoconjugate subunits and the detecting reagents allowed high-avidity binding to immobilized multivalent fluoresceinated glycoconjugate to be established. FIG. 5 shows the binding matches between spores (target entities) and immobilized glycoconjugates (ligands). In summary, Gal α 1-3 GalNAc α-PAA-flu, Gal β 1-4 Glc β-PAA-flu bound to B. cereus spores. Fuc α 1-4 GlcNAc β-PAA-flu, Fuc α1-3 GlcNAc β-PAA-flu bound to B. thuringiensis spores. GlcNAc β 1-4 GlcNAc β-PAA-flu, Gal β1-3 Gal β-PAA-flu, bound to B. subtilis spores. Gal β1-3 GalNAc β-PAA-flu , Gal β1-3GalNAc α-PAA-flu to bound B. pumilus spores.

These results demonstrate that carbohydrate conjugates can selectively detect spores which have glucoconjugate epitopes within a native spore's exterior. With the exemplary embodiment, most of the detected carbohydrate-carbohydrate binding interactions were consistent with monosaccharide specificities found in FACE results. The inventors believe that carbohydrates located on the spores' surface created multivalent displays that bound avidly and specifically to carbohydrate-binding epitopes. The inventors further believe that the experimental evidence also directly implicates complex carbohydrates in the recognition processes, including adhesion between cells, adhesion of cells to the extracellular matrix, and specific recognition of cells by one another.

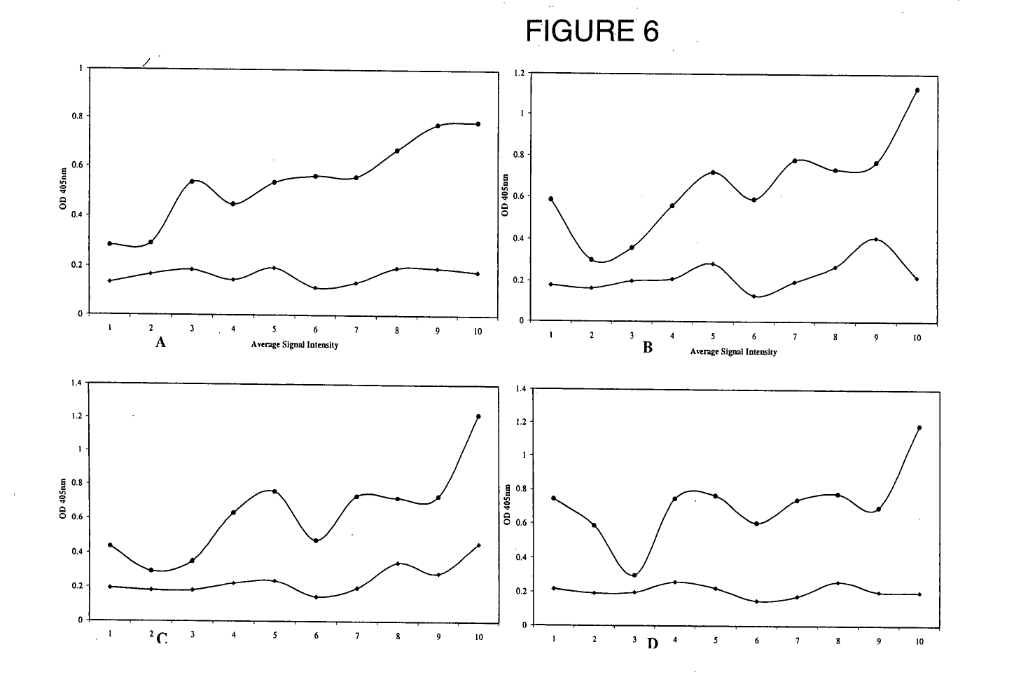

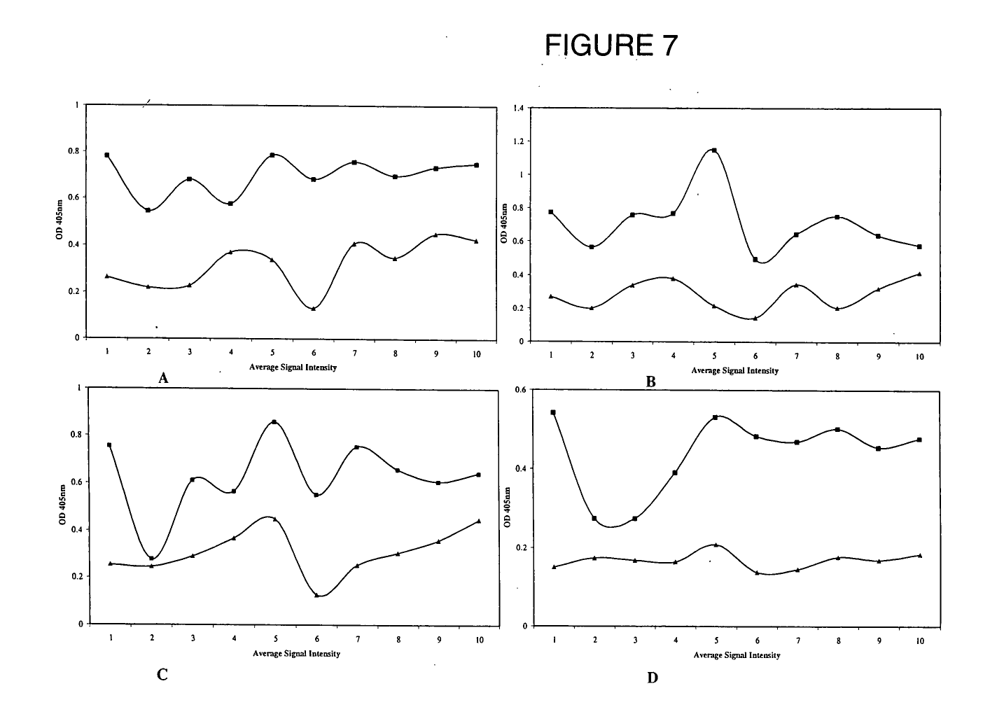

To examine the dose-dependency of glycoconjugates on spores recognition, solutions of glycoconjugates were prepared according manufacture procedure and then serially ×10, ×100, ×1000 times diluted into in PBS/0.2% NaH₃ buffer. Typical binding curves of target/s recognition are shown in the graphs of FIG. 6 and 7. The use of diluted glycoconjugates as a capture reagent allowed B. subtilis to be recognized and distinguished from B. cereus the spores as shown at the graph of FIG. 6. We evaluated glycoconjugate platform to discriminate related Bacilli species including B. thuringiensis and B. pumilus spores (See the graph of FIG. 7). Taken together, serially diluted glycoconjugates were able to recognize and distinguish studied spores (See B-D on the graphs of FIGS. 6 and 7).

§ 5.5 Alternatives and Refinements

Although some of the embodiments described above include sensors to detect biological molecules using polyvalent carbohydrate-carbohydrate interactions, the present invention is not limited to “polyvalent carbohydrate-carbohydrate interactions”. For example, in addition to polyvalent interactions (e.g., sugar linked to polymer), the present invention may also use monovalent (e.g., only one sugar) and/or multivalent (e.g., many different sugars on polymer) interactions. Furthermore, sugar conjugates can also interact with other TAMPs, such as proteins for example, on the surface of a target entity.

Although some embodiments of the present invention used spore surface analysis for identifying the glycoconjugate(s), which were used to select a binding partner, the present invention is not limited to the spore surface analysis to identify TAMPs (e.g., glyconconjugate(s)) and select binding partners. For example, TAMPs (e.g., glycoconjugate(s)) can also be identified from a random set of glycomolecules in a screening experiment for the specific binding. The carbohydrate binding partner component in the ligand conjugate can also be selected using high throughput methods, such as flow cytometry, or other screening methods for selective binding.

The sugar molecules identified can be conjugated with covalent linking, such as ester or amide bonding, or through ionic or other non-covalent interactions with the conjugating molecules which can be small molecular bifunctional or multifunctional linkers, or tethers, or dendrimers of various generations or synthetic or natural macromolecules of various molecular weights.

Although some embodiments of the present invention use fluorescent labeled glycoconjugates as model systems to evaluate the mechanism of Bacillus spore recognition, any other type of transduction mechanism can be applied for the detection of the specific binding. However, other transduction techniques (such as acoustic, optical, electrical, electrochemical, or mass based) may be used. Accordingly, although some embodiments of the present invention used an ELISA plate as a support surface, other support surfaces are possible. For example, the support surface could be: a plate for surface acoustic wave measurement; a quartz crystal microbalance support surface, or another surface which is sensitive to changes in mass; a support surface on any electrochemical device such as on ion sensitive electrode or ion selective field effect transistor; a support surface on light emitting or otherwise optical active surface; etc.

The amount of glycoconjugate, as well as spores, may be decreased to develop a miniature platform. At the present time we used 20 μl multivalent glycoconjugate and 7.5 μl spores.

### § 6. CONCLUSIONS

Products, apparatus and methods consistent with the principles of the present invention can afford several advantages. First, the glycoconjugate sensor's output may be quantitative. A chromogenic reaction product, for example, may be quantitatively determined using a plate reader upon completion of the immuno- and enzymatic reactions.

The present invention has also dramatically increased the potential of rapidly determining the presence of specific carbohydrate epitopes when the ELISA sensor is used. Primarily, this is because of the high surface area to volume ratio in the immunosorbent. Further, the U-shaped wells of a microtiter plate ensure improved contact between the sample and solid-phase glycoconjugates, thus producing an increased antigen-antibody encounter rate. Thus, immunobinding is quantitatively achieved during the relatively short time of immunoassays.

Additionally, glycoconjugates were observed to interact with certain spores' epitopes. Thus, the present invention may be valuable in discovering unexpected but biologically relevant bacterial species. Detecting carbohydrate-binding epitopes is considered adequate for this type of application since most pathogens possess unique cell-surface carbohydrates.

A major advantage of apparatus, products and methods consistent with the principles of the present invention is their glycoconjugate-specificity and selectivity to spore species, as well as the accuracy of detecting as few as 2.2×10⁵ bacterial spores in a single sample of 7.5 μl. The present data, suggesting means for its potential use, is addressing either the carbohydrate microarray library or the components in suspension, or even both. The present invention may also help expand information concerning multiple aspects of carbohydrate-carbohydrate recognition in applications such as detection of bacterial spores.

Furthermore, methods consistent with the present invention may be used to construct glycoconjugate devices to be used as “test strip” products. Such products could be easily transported to a remote site and later analyzed to test for the presence of spores or other biological entities. Under such methods, data could be collected on location using test strip products (e.g., consumable, disposable glycoconjugate products) and the test strip products brought to a laboratory for analysis.

The present invention can be used to produce other sensors in which other surfaces may be used as the sensor's support surface. Carbohydrates may be appended to polymers and used as the substrate coating the support surface. Colorimetry, or other detection methods may be used to detect carbohydrate-carbohydrate binding. The serially diluted glycoconjugates can recognize and distinguish bacterial spores. Patterns of binding curves may be used as an algorithm for recognition of bacterial species.

What is claimed is:

1. An apparatus for detecting a biological target, the apparatus comprising:
a) a support surface;
b) glycopolymers, able to bind with surface target-associated molecular patterns of the target, coating the support surface; and
c) transduction means for detecting a binding event between the glycopolymers and the glycoconjugates.

2. The apparatus of claim 1 wherein the support surface is selected from a group consisting of (A) an ELISA plate, (B) a plate for surface acoustic wave measurement, (C) a surface on a quartz crystal microbalance, (D) a surface on a transduction means sensitive to changes in mass, (E) a surface on an electrochemical device, (F) a surface on an ion sensitive electrode, (G) a surface on an ion selective field effect transistor, (H) a surface on a light emitting surface, and (I) a surface on an optically active surface.

3. The apparatus of claim 1 wherein the glycopolymers are carbohydrates appended to polymers.

4. The apparatus of claim 1 wherein the glycopolymers are sugar molecules conjugated with covalent linking.

5. The apparatus of claim 4 wherein the covalent linking uses ester or amide bonding.

6. The apparatus of claim 1 wherein the glycopolymers are sugar molecules linked, through ionic or other non-covalent interactions, with conjugating molecules.

7. The apparatus of claim 6 wherein the conjugating molecules are selected from a group of conjugating molecules consisting of (A) small molecular bifunctional linkers, (B) small molecular multifunctional linkers, (C) tethers, (D) dendrimers of various generations, (E) synthetic macromolecules, and (F) natural macromolecules.

8. The apparatus of claim 3, wherein the polymers are polyacrylamide (PAA).

9. The apparatus of claim 1 wherein the glycopolymers are fluorescent.

10. The apparatus of claim 1 wherein the glycopolymers are multivalent.

11. The apparatus of claim 1 wherein the glycopolymers are monovalent.

12. The apparatus of claim 1 wherein the glycopolymers are polyvalent.

13. The apparatus of claim 1 wherein the means for detecting a binding event is antibody color detection.

14. The apparatus of claim 1 wherein the biological target is a bacterial spore.

15. The apparatus of claim 1 wherein the biological target is Bacillus cereus spores.

16. The apparatus of claim 15 wherein the target-associated molecular patterns include at least two of Gal α 1-3 GalNAc α-PAA-flu, Gal β 1-4 Glc β-PAA-flu.

17. The apparatus of claim 1 wherein the target is Bacillus thuringiensis spores.

18. The apparatus of claim 17 wherein the target-associated molecular patterns include at least two of Fuc α 1-4 GlcNAc β-PAA-flu, Fuc α1-3 GlcNAc β-PAA-flu.

19. The apparatus of claim 1 wherein the target is Bacillus subtilis spores.

20. The apparatus of claim 19 wherein the target-associated molecular patterns at least two of GlcNAc β 1-4 GlcNAc β-PAA-flu, Gal β1-3 Gal β-PAA-flu.

21. The apparatus of claim 1 wherein the target is Bacillus pumilus spores.

22. The apparatus of claim 21 wherein the target-associated molecular patterns include at least two of Gal β1-3GalNAc β-PAA-flu, Gal α 1-3 GalNAc α-PAA-flu.

23. A method for fabricating a glycoconjugate sensor for sensing a target, the method comprising:
a) coating a support surface with glycopolymers able to bind with target-associated molecular patterns on a surface of the target; and
b) incorporating means to detect a binding event between target-associated molecular patterns on the surface of the target and the glycopolymers.

24. The method of claim 23 wherein the surface target-associated molecular patterns are identified by fluorophore assisted carbohydrate electrophoresis analysis.

25. The method of claim 24 further comprising identifying carbohydrate binding partners able to bind with the target-associated molecular patterns.

26. The method of claim 23 wherein the support surface is an ELISA plate.

27. The method of claim 23 wherein the glycopolymers are carbohydrates appended to polymers, and wherein the polymers are polyacrylamide (PAA).

28. The method of claim 23 wherein the glycopolymers are fluorescent.

29. The method of claim 23 wherein the glycopolymers are multivalent.

30. The method of claim 23 wherein the glycopolymers are monovalent.

31. The method of claim 23 wherein the glycopolymers are polyvalent.

32. The method of claim 23 wherein the support surface is an ELISA plate, and wherein the act of coating a support surface includes
i) coating wells of an ELISA plate with glycopolymers;
ii) incubating the coated plate;
iii) washing the incubated coated plate;
iv) blocking the washed, incubated, coated plate; and
v) incubating the blocked plate.

33. The method of claim 23 wherein the target is Bacillus cereus spores.

34. The method of claim 33 wherein the glycopolymers include at least two of Gal α 1-3 GalNAc α-PAA-flu, Gal β1-4 Glc β-PAA-flu.

35. The method of claim 23 wherein the target is Bacillus thuringiensis spores.

36. The method of claim 35 wherein the glycopolymers include at least two of Fuc α 1-4 GlcNAc β-PAA-flu, Fuc α1-3 GlcNAc β-PAA-flu.

37. The method of claim 23 wherein the target is Bacillus subtilis spores.

38. The method of claim 37 wherein the glycopolymers include at least two of GlcNAc β 1-4 GlcNAc β-PAA-flu, Gal β1-3 Gal β-PAA-flu.

39. The method of claim 23 wherein the target is Bacillus pumilus spores.

40. The method of claim 39 wherein the glycopolymers include at least two Gal β1-3 GalNAc β-PAA-flu, Gal α 1-3 GalNAc α-PAA-flu.

41. A method for detecting target entities in solution, the method comprising:
a) exposing a sensor coated with glycopolymer substrate to a solution containing targets with target-associated molecular patterns on their surfaces;
b) allowing specific binding between the target-associated molecular patterns on the surface of the target and glycopolymers of the sensor to occur; and
c) identifying specific binding, if any, between the target-associated molecular patterns on the surfaces of the targets and the glycopolymers of the sensor.

42. The method of claim 41 wherein the act of identifying specific binding is based on a calorimetric reaction.

43. The method of claim 42 wherein the calorimetric reaction is quantifiable by spectrophotometric analysis.

44. The method of claim 41 wherein the sensor is an ELISA glycoconjugate sensor.

45. The method of claim 41 wherein the specific binding is a carbohydrate interaction with the target.

46. A product for recognizing target entities in solution, the product comprising:
a) a support surface;
b) glycopolymers, able to bind with target-associated molecular patterns on a surface of the target, coating the support surface.

47. The product of claim 46 wherein the support surface is an ELISA plate.

48. The product of claim 46 wherein the glycopolymers are carbohydrates appended to polymers.

49. The product of claim 48 wherein the polymers are polyacrylamide (PAA).

50. The product of claim 46 wherein the glycopolymers are fluorescent.

51. The product of claim 46 wherein the glycopolymers are multivalent.

52. A system for detecting a biological target is solution, the system comprising:
a) a solution including glycopolymers, able to bind with target-associated molecular patterns on a surface of the target; and
b) transduction means for detecting a binding event between the glycopolymers and the target-associated molecular patterns.

53. The system of claim 52 wherein the glycopolymers are fluorescent.

54. The system of claim 52 wherein the glycopolymers are multivalent.

55. The system of claim 52 wherein the glycopolymers are monovalent.

56. The system of claim 52 wherein the glycopolymers are polyvalent.

57. The system of claim 52 wherein the biological target is a bacterial spore.

58. The system of claim 52 wherein the biological target is Bacillus cereus spores.

59. The system of claim 58 wherein the glycopolymers include at least two of Gal α 1-3 GalNAc α-PAA-flu, Gal β 1-4 Glc β-PAA-flu.

60. The system of claim 52 wherein the target is Bacillus thuringiensis spores.

61. The system of claim 60 wherein the glycopolymers include at least two of Fuc α 1-4 GlcNAc β-PAA-flu, Fuc α1-3 GlcNAc β-PAA-flu.

62. The system of claim 52 wherein the target is Bacillus subtilis spores.

63. The system of claim 62 wherein the glycopolymers include at least two of GlcNAc β 1-4 GlcNAc β-PAA-flu, Gal β1-3 Gal β-PAA-flu.

64. The system of claim 52 wherein the target is Bacillus pumilus spores.

65. The system of claim 64 wherein the glycopolymers include at least two of Gal β1-3GalNAc β-PAA-flu, Gal α1-3 GalNAc α-PAA-flu.

66. The method of claim 41 further comprising:
d) generating a binding curve from identified specific bindings, if any, between the target-associated molecular patterns on the surface of the target and the glycopolymers of the sensor; and
e) identifying the target using the generated binding curve.

67. The method of claim 41 wherein the sensor coated with glycopolymer substrate includes a number of areas, each area having a glycopolymer with a different concentration of glycoconjugates.

68. The method of claim 41 wherein the sensor coated with glycopolymer substrate includes a number of areas, each area having a glycopolymer with a serially diluted concentration of glycoconjugates.

69. The apparatus of claim 1 wherein the target-associated molecular patterns are glycoconjugates.

70. The method of claim 23 wherein the target-associated molecular patterns are glycoconjugates.

71. The method of claim 41 wherein the target-associated molecular patterns are glycoconjugates.

72. The product of claim 46 wherein the target-associated molecular patterns are glycoconjugates.

73. The system of claim 52 wherein the target-associated molecular patterns are glycoconjugates.
